(19) United States
(12) Patent Application Publication
(10) Pub. No.: US 2024/0219379 A1
(43) Pub. Date: Jul. 4, 2024

(54) IMMUNOASSAY DEVICE AND METHOD

(71) Applicant: IDEXX Laboratories, Inc., Westbrook, ME (US)

(72) Inventors: Murthy Yerramilli, Westbrook, ME (US); Dustin Wride, Westbrook, ME (US); Hongzhi Xie, Westbrook, ME (US); William Foster, Westbrook, ME (US); Jennifer Gagner, Westbrook, ME (US)

(21) Appl. No.: 18/390,970

(22) Filed: Dec. 20, 2023

Related U.S. Application Data
(60) Provisional application No. 63/436,198, filed on Dec. 30, 2022.

(51) Int. Cl. G01N 33/53; G01N 33/543

(52) U.S. Cl. CPC G01N 33/5308; G01N 33/54388; G01N 2333/4353; G01N 2469/10

(57) ABSTRACT

Immunoassay devices, kits and methods for determining a first analyte in a sample. The device a membrane that supports lateral flow of a liquid, and a dried first detection reagent comprising a first gold nanoparticle having bound thereto (i) a first binding partner for the first analyte, and (ii) a first label, wherein the dried first detection reagent is dried on the membrane and solubilizable by a liquid. Methods using the device to determine analytes in liquid samples.

### CROSS-REFERENCE TO RELATED APPLICATION

This application claims the benefit of U.S. provisional application Ser. No. 63/436,198, filed Dec. 30, 2022, which is incorporated by reference in its entirety.

### FIELD

The present disclosure relates generally to the immunoassay devices and associated reagents for determining the presence of analytes in liquid samples.

### BACKGROUND

Many immunoassays devices rely on liquid flow of sample and reagents on a membrane. Often, mixing of samples and reagents for the assay is completed before application of the mixture to the device. This requires multiple reagent containers and additional steps by an operator. In addition, liquid reagents may have a short shelf-life.

Accordingly, the present device and reagents for lateral flow assays that minimize operator involvement and provide for increased storage stability provide benefits over the prior technology.

### SUMMARY

In one aspect, the disclosure is directed to an immunoassay device for determining a first analyte in a sample. The device includes a membrane that supports lateral flow of a liquid, and dried first detection reagent including a first gold nanoparticle having bound thereto (i) a first binding partner for the first analyte, and (ii) a first label, wherein the dried first reagent is dried on the membrane and solubilizable by a liquid. The membrane may include sample application zone and a detection zone, and the dried first detection reagent may be dried in the sample application zone. The device may also include a reagent zone between the sample application zone and the detection zone, wherein the dried first detection reagent is dried in the reagent zone. In addition, the device may include a first immobilized capture reagent in the detection zone including a second binding partner for the analyte or an analyte analog non-diffusively bound to the membrane. The first and/or second binding partner for the first analyte is an anti-analyte antibody.

In one aspect of the disclosure, the reagent zone includes a blocking reagent that prevents chemical interaction between the detection reagent and the membrane.

In another aspect, the disclosure includes a housing supporting the membrane and including a container including a wash reagent and/or and a container including the substrate.

In another aspect of the disclosure wherein the device has a shelf life of over at least about 12 months at room temperature.

In yet another aspect, the device of the disclosure may include a dried second detection reagent in the sample application zone or the reagent zone including a second gold nanoparticle having bound thereto (i) a third binding partner for a second analyte, and (ii) a second label, wherein the second dried reagent is solulizable by a liquid. The second immobilized capture reagent in the detection zone includes a fourth binding partner for the second analyte, wherein the first gold particle and the second gold particle are the same, and the first label and the second label may be the same or different. In addition, the first label and the second label may be the same, and the first immobilized capture reagent and the second immobilized capture reagent are immobilized in distinct regions of the detection zone.

In another aspect, the disclosure is directed to a method of determining the presence or amount of an analyte in a sample. The method includes adding a sample suspected of containing the analyte to the sample application zone of the device of the disclosure, allowing the sample to migrate by lateral flow through the reagent zone and the detection zone, and detecting the presence or amount of the label in the detection zone, wherein the presence or amount of the label provides a signal associated with the presence or amount of the analyte in the sample.

### BRIEF DESCRIPTION OF FIGURES

The accompanying drawings, which are included to provide a further understanding of the disclosure, are incorporated in and constitute a part of this specification, illustrate embodiments of the disclosure, and together with the detailed description serve to explain the principles of the disclosure. No attempt is made to show structural details of the disclosure in more detail than may be necessary for a fundamental understanding of the disclosure and various ways in which it may be practiced.

FIG. 1 is a variability chart showing higher sensitivity of antigen detection with HRP/Ab:GNP over Ab:GNP. Hookworm recombinant antigen was tested on a lateral flow immunoassay device using HRP/Ab:GNP (HRP) or Ab:GNP (GNP) as detection agents. The y-axis is the sample spot density minus background spot density value. The x-axis shows the anti-Hookworm coated gold nanoparticle (GNP) and the Horseradish peroxidase/anti-Hookworm coated gold nanoparticle (HRP) in the absence (0 ng/ml) and presence (180 ng/ml) of the Hookworm recombinant antigen used in the sample diluent. The data was plotted from the experiment pictured in FIG. 3.

FIG. 2 shows images of the lateral flow immunoassay devices using the anti-Hookworm Ab:GNP (upper) and HRP/Anti-Hookworm Ab:GNP (lower) detection reagent, after reaction with 180 ng/ml (left) and 0 ng/ml (right) of Hookworm recombinant antigen sample. A slight red spot on the upper left indicates a positive result with Ab:GNP (not clearly visible in grey scale reproduction of the image). A blue circle on the lower left indicates a positive result with HRP/Ab:GNP (shown in grey scale).

FIG. 3 is a variability chart showing that HRP/Ab:GNP in a dried down state (liquid reagent was dried in a tube and mixed with the antigen sample solution upon experiment) did not lose its sensitivity over the liquid state (liquid reagent was mixed with the antigen sample solution upon experiment). The y-axis is the sample spot density minus background spot density value. The x-axis shows the Dried or Liquid detection reagent (conjugate condition), and the concentration of the Hookworm recombinant antigen used in the sample diluent. p/n=positive/negative.

FIG. 4 shows a representation of a lateral flow immunoassay device with HRP/Anti-Hookworm Ab:GNP dried on a membrane and stored onboard (stripe). As the sample solution flowed from the sample cup (left circle to the stripe) to a detection zone (right of the stripe), the dried HRP/Ab:GNP can be reconstituted in liquid form.

FIG. 5 is a variability chart showing the functionality of HRP/Anti-Hookworm Ab:GNP in membrane-dried state and stored inside the immunoassay device as shown in FIG. 4. The y-axis is the sample spot density minus background spot density value. The x-axis shows the Dried or Liquid detection reagent (Conjugate condition), and the concentration of the Hookworm recombinant antigen used in the sample diluent. p/n=positive/negative.

FIGS. 6A and 6B is a variability chart showing differentiation of target antigen with both recombinant antigen as well as real samples. Hookworm (HW), Roundworm (RW) and Whipworm (WW) reagents were multiplexed, dried onto a matrix as described herein and used to test rAG (FIG. 6A) and fecal (real) (FIG. 6B) samples. Differentiation of target antigen was shown with both recombinant antigen as well as real samples. p/n=positive/negative.

### DESCRIPTION

The disclosure is directed to an immunoassay device including reagents for use in the determination of analytes in liquid samples. The device and reagents provide for increased assay sensitivity, reduced operator workflow, and improved stability of the device and reagents. The device provides a lateral flow membrane with a non-diffusively bound nanoparticle-based reagent that includes both a binding partner for the analyte in a sample and a label that provides a signal for indication of such binding.

Before describing the present invention in detail, a number of terms will be defined. As used herein, the singular forms “a,” “an”, and “the” include plural referents unless the context clearly dictates otherwise.

The term “analyte,” as used herein, generally refers to the substance, or set of substances in a sample that are detected and/or measured. An analyte may be, for example, a protein, a glycoprotein, a saccharide, a polysaccharide, an amino acid, a substituted amino acid, a methylated amino acid, a hormone, an antibiotic, a nucleic acid, a metabolite, or a derivative of any of the foregoing.

The term “analog,” as used herein, generally refers to a compound in which one or more individual atoms have been replaced with a different atom(s) or with a different functional group(s) that provide a means to join the analyte to another moiety, such as a label or solid matrix. For example, a means to join the analyte to another moiety may be a linker. An analog may compete with the analyte for a receptor. In particular, the analyte analog can bind to an antibody in a manner similar to the analyte. Because covalent binding of the analyte to a matrix or another molecule is often accomplished through the use of an analyte analog, the disclosure herein of simply “the analyte” attached to or conjugated to a matrix or another molecule includes the use of an analyte analog to accomplish such covalent attachment or conjugation as would be readily understood by one of ordinary skill in the art of immunoassays.

The term “antibody,” as used herein, generally refers to a glycoprotein produced by B lymphocyte cells in response to exposure to an antigen and binds specifically to that antigen. The term “antibody” is used in its broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

The term “antibody fragment,” as used herein, refers to a portion of a full-length antibody, generally the antigen binding or variable domain thereof. Specifically, for example, antibody fragments may include Fab, Fab′, F(ab′)2, and Fv fragments; diabodies; linear antibodies; single-chain antibody molecules; and multispecific antibodies from antibody fragments.

The term “antigen,” as used herein, generally refers to a substance that is capable, under appropriate conditions, of reacting with an antibody specific for the antigen.

The term “animal” as used herein, generally refers to any animal, e.g., a human, or a non-human animal including companion animals, livestock, and animals in the wild.

The term “sample,” as used herein, generally refers to a sample of tissue or fluid from a human or animal including, but not limited to whole blood, plasma, serum, spinal fluid, lymph fluid, abdominal fluid (ascites), the external sections of skin, respiratory, intestinal and genitourinary tracts, tears, saliva, urine, blood cells, tumors, organs, tissue, feces and sample of in vitro cell culture constituents. Many such samples require processing prior to analysis. Sample includes both raw samples and/or processed samples.

The term “immunoassay,” as used herein, generally refers to a test that employs antibody and antigen complexes to generate a measurable response. An “antibody:antigen complex” may be used interchangeably with the term “immuno-complex.” Immunoassays, in general, include noncompetitive immunoassays, competitive immunoassays, homogeneous immunoassays, and heterogeneous immunoassays. In “competitive immunoassays,” unlabeled analyte (or antigen) in the test sample is measured by its ability to compete with labeled antigen in the immunoassay. The unlabeled antigen blocks the ability of the labeled antigen to bind because the binding site on the antibody is already occupied. In “competitive immunoassays,” the amount of antigen present in the test sample is inversely related to the amount of signal generated from the label. Conversely, in “noncompetitive immunoassays,” also known as “sandwich” immunoassays, the analyte is bound between two highly specific antibody reagents to form a complex and the amount of antigen is directly proportional to the amount of signal associated with the complex. Immunoassays that require separation of bound antibody:antigen complexes are generally referred to as “heterogeneous immunoassays,” and immunoassays that do not require separation of antibody:antigen complexes are generally referred to as “homogeneous immunoassays.” One of skill in the art would readily understand the various immunoassay formats.

The term “label,” as used herein, refers to a detectable compound, which can be conjugated directly or indirectly (e.g., via covalent or non-covalent means, alone or encapsulated) to an antibody or analogs of the disclosure. The label may be detectable by itself (e.g., chemiluminescent dye, fluorescent labels) or, in the case of an enzymatic label, may catalyze chemical alteration of a substrate compound or composition which is detectable (e.g., enzymes such as horseradish peroxidase, alkaline phosphatase, and the like). The label employed in the current disclosure could be, but is not limited to: alkaline phosphatase; glucose-6-phosphate dehydrogenase (“G6PDH”); horse radish peroxidase (HRP); chemiluminescers such as isoluminol, fluorescers such as fluorescein and rhodamine compounds; ribozymes; dyes and time resolved fluorescent labels (e.g., a lanthanide chelate). The label produces a signal that may be detected by means such as detection of electromagnetic radiation or direct visualization, and that can optionally be measured.

The terms “membrane” or “matrix” as used herein, refer to a non-aqueous matrix to which the components of an immunoassay, including the reagents of the disclosure to be diffusively or non-diffusively bound. Examples of membranes and matrices include supports formed partially or entirely of glass (e.g., controlled pore glass), synthetic and natural polymers, polysaccharides (e.g., agarose), polyacrylamides, polystyrene, polyvinyl alcohols and silicones that are formed in to chromatographic that will allow reagents that become bound to be washed or separated from unbound materials. In some embodiments, the membranes or matrices can be porous.

The term “about,” as used herein means ±10%, preferably ±5%, more preferably, 2%, and most preferably ±1%.

The term “particle” or “particles” in connection with the disclosure include, for example, gold nanoparticles and particles of latex, polystyrene, or of other support materials such as silica, agarose, ceramics, glass, polyacrylamides, polymethyl methacrylates, carboxylate modified latex, melamine, and Sepharose. The particles will vary in size from about 0.1 microns to about 100 microns, for example about 0.1, 0.5, 1.0, 5, 10, 20, 30, 40 50, 60, 70, 80 90 or 100 microns. In particular, useful commercially available materials include carboxylate modified latex, cyanogen bromide activated Sepharose beads, fused silica particles, isothiocyanate glass, polystyrene, and carboxylate monodisperse microspheres.

The particles, other than the gold nanoparticles, may be magnetic or paramagnetic. Particles suitable for use in the present disclosure are capable of attachment to other substances such as derivatives, linker molecules or proteins. The capability of the particles to be attached to other substances can result from the particle material as well as from any surface modifications or functional groups added to the particle. The particles can be functionalized or be capable of becoming functionalized in order to covalently or non-covalently attach proteins, linker molecules or derivatives as described herein. Suitable functional groups include, for example, amine, biotin, streptavidin, avidin, protein A, sulfhydryl, hydroxyl and carboxyl.

Gold nanoparticles are well known for use in immunoassays and may be surface modified to minimize aggregation and allow them to be coated with antibodies or other molecules. In various embodiments, particle range in size from about 10 nm to about 200 nm, for example about 10, 20, 30, 40, 50, 60, 70, 80, 90, 100, 120, 140, 160, 180 or 200 nm. In various embodiments, the particles size may be about 10-50 nm or 20-40 nm.

All patent references identified herein are incorporated by reference herein in their entirety.

Turning now to the various aspects of the disclosure, an immunoassay device for determining an analyte in a sample is described herein. The device includes a membrane that supports lateral flow of a liquid, and a dried detection reagent that is dried on the membrane so that it can be solubilized by the sample or a liquid reagent that has been applied to the membrane. The reagent includes gold nanoparticle that has bound thereto a binding partner for the analyte and label. Drying of the detection reagent on the membrane relieves the operator of the separate step of adding the reagent to the device or the sample prior to adding the sample to the device. In addition, the membrane with the dried reagent provides for enhanced storage longevity that exceeds the shelf-life of liquid reagents.

In some embodiments of the disclosure, the membrane supports lateral flow of the sample and, optionally, liquid reagents that are applied to the membrane. In some embodiments, the membrane includes a sample application zone, a reagent zone, and a detection zone. Sample applied to the device in the sample application zone migrates across the membrane through the reagent zone and towards the detection zone. The sample solubilizes the detection reagent that is dried in the detection zone and transports the detection reagent along with the analyte in the sample, if any, towards the detection zone. The device may be configured so that the sample application zone and the reagent zone are contiguous or are partially or completely overlapping.

Lateral flow devices, associated membranes and dried reagents are generally known. For instance, the membrane can be one of many known materials that support lateral flow of liquids. The suitable materials include fibrous mats composed of synthetic or natural fibers (e.g., glass or cellulose-based materials or thermoplastic polymers, such as, polyethylene, polypropylene, or polyester); sintered structures composed of particulate materials (e.g., glass or various thermoplastic polymers); or cast membrane films composed of nitrocellulose, nylon, polysulfone or the like (generally synthetic in nature). The matrix may also be composed of sintered, fine particles of polyethylene, commonly known as porous polyethylene, such as sintered polyethylene beads. As a non-limiting examples, such material may have a density of between 0.35 and 0.55 grams per cubic centimeter, a pore size of between 5 and 40 microns, and a void volume of between 40 and 60 percent. Particulate polyethylene composed of cross-linked or ultra-high molecular weight polyethylene may also be used. An example matrix includes 1015 micron porous polyethylene from Chromex Corporation FN #38-244-1 (Brooklyn, N.Y.) and FUSION 5™ matrix available from Whatman, Inc., USA.

Gold nanoparticles have been used in lateral flow assays, often as labels themselves. In the various aspects of the disclosure, however, the gold nanoparticles provide a soluble carrier upon which a separate label (or component of a signal producing system such as an enzyme) and a binding partner for the analyte are attached. Accordingly, in various aspects of the disclosure, the label is not the gold nanoparticle.

The label should be detectable visually or by spectrometry. For example, the label may be an enzyme that provides a visual signal upon contact with a substrate. An example enzyme is horseradish peroxidase (HRP) for which substrates are generally known.

In this embodiment, the detection reagent of the disclosure may include, for example, an anti-analyte antibody bound to a gold nanoparticle and separately HRP bound to the gold nanoparticle. The reagent (abbreviated herein as “HRP/Ab:GNP”) may be dried in a reagent zone of the device as described herein. The Examples below compare the performance of embodiments of HRP/Ab:GNP (using HRP as the label) to detection reagents that use the gold nanoparticle as the label (abbreviated herein as “Ab:GNP”). As shown in the Examples below, embodiments using HRP/Ab:GNP provide an increased assay sensitivity over embodiments using Ab:GNP.

In one aspect, a blocking reagent may be applied to the reagent zone and allowed to dry prior to the application of the HRP/Ab:GNP to the reagent zone to prevent chemical interaction between the membrane material and the HRP/Ab:GNP. The blocking reagent may include, for example, a polymeric material (e.g., PVP) that provides a neutral surface and prevents degradation of the HRP/Ab:GNP in the presence of some membrane materials. The blocking of the membrane also enhances storage stability of the device.

In one aspect, the immunoassay device has an immobilized capture reagent in the detection zone of the device. For example, the immobilized capture reagent can be an anti-analyte antibody that binds the analyte in the sample. The antibody of the capture reagent may be directly attached to the membrane in the detection zone or may be attached to a particle (e.g., a latex particle) that is immobilized in the detection zone. The disclosure includes immunoassay devices for conducting assays using a sandwich format wherein the analyte is captured by both the antibody immobilized in the detection zone and the antibody of the HRP/Ab:GNP. Additionally, assays using a competitive format may be used in which situation an analog for the analyte may be immobilized in the detection zone that completes with analyte in the sample for binding to the detection reagent.

In aspects of the disclosure, the lateral flow membrane of the device is supported in a housing for supporting the membrane and may include a container holding a liquid wash reagent. In addition, the housing may include a container holding a liquid substrate that includes a substrate for the label when it is an enzyme (a “substrate reagent”). Embodiments of a housing including onboard wash and substrate reagents are shown, for example, in U.S. Pat. No. 5,627,010 and are sold under the SNAP® brand by IDEXX Laboratories, Inc. Briefly, upon addition of the sample to such a device, the sample migrates past a reagent zone where it contacts the dried reagent of the disclosure. The sample then migrates to the detection zone wherein the analyte, if present, is sandwiched by a binding partner immobilized in the detection zone and the binding partner attached to the gold nanoparticle. An operator can activate the device to release the wash reagent and the substrate reagent onto the lateral flow membrane such that migration of the wash reagent removes unbound reactants from the detection zone and the substrate produces a signal by reacting with the enzyme label attached to the gold particle. The signal can be read by the operator by visual or spectrophotometric means. In some embodiments, the wash and substrate reagents are the same reagent. If the label does not require a substrate for detection (e.g., a fluorescent label), the device would not require the substrate reagent.

In addition to improving assay sensitivity for antigen targets to be detected in a sample by using an HRP/Ab:GNP reagent dried down on a membrane according to the disclosure, Applicants also determined that the device provides for increased storage stability for the device when compared to liquid detection reagents. In particular, the storage stable device has a shelf life of over about 12 months, for example 15 months, 18 months, 21 months, or 24 months at room temperature.

Another aspect of the disclosure is directed to a multiplex device that has the ability to determine more than one analyte in the sample. In this aspect, a dried second detection reagent in the reagent zone includes a second gold nanoparticle having bound thereto (i) a binding partner for a second analyte, and (ii) a second label. Like a first detection reagent, the second dried reagent is solubilizable by a liquid. A second immobilized capture reagent in the detection zone includes a binding partner for the second analyte. In this aspect, the first gold particle and the second gold particle may be the same or different and the first label and the second label may be the same or different. The detection zone may have distinct areas for capturing each of the two antigens in the sample so that the labels, even if the same, can be distinguished and associated with the correct analyte.

In further aspects, the device is capable of detecting a third, a fourth or more analytes in the sample. In such aspects, the appropriate capture reagents should be immobilized in the detection zone while the labels, attached to the Ab:GNP should be distinguishable either by location of the immobilized capture reagent or by differentiation in the labels themselves.

In yet another aspect, the disclosure is directed to a method for determining the presence or amount of an analyte in a sample. The method includes adding a sample suspected of containing the analyte to the sample application zone of a device according to the disclosure and allowing the sample to migrate by lateral flow through the reagent zone and the detection zone. An operator can detect the presence or amount of the label in the detection zone, wherein the presence or amount of the label provides a signal associated with the presence or amount of the analyte in the sample. As noted above, the device may include a housing supporting the membrane and may include a container(s) for a wash reagent and a substrate reagent such that, upon activation of the device, the wash reagent washes the detection zone to remove unbound reactants. The substrate for the label can provide a detectable signal if the sample contains an analyte.

The device of the disclosure allows for a method that uses the liquid sample applied directly to the device with no need for any reagent kits or cups. As noted above, the sample may be unprocessed (e.g. whole blood, saliva or urine) or may be processed to render the sample in liquid form (e.g., stool sample). Non-liquid samples often must be liquefied in a buffer or other diluent and subject to filtration, and/or centrifugation to avoid clogging the matrix with particulate matter once the sample is applied to the membrane.

In some embodiments, the disclosure is directed to using one or more reagents of the disclosure for detecting fecal antigens including Hookworm, Roundworm and Whipworm in a stool sample that has been processed for application to the device in liquid form. In example embodiments, the detection reagent is a detection antibody for one of the fecal antigens (described in Examples below) co-adsorbed with the HRP enzyme on a 40 nm gold nanoparticle. The HRP/Ab:GNP reagent can be deposited onto a lateral flow membrane of an assay device of the disclosure to allow for mixing with the patient sample on the device without the need for mixing steps outside of the device.

### EXAMPLES

### Description of Assays

As a model system, the detection and capture reagents were tested on a sintered matrix immune assay device format (SNAP® device, IDEXX Laboratories, Inc.) that utilizes a reversible flow chromatographic binding assay (U.S. Pat. No. 5,726,010) to determine the sensitivity and specificity of the fecal antigen immunoassay using the HRP/Ab:GNP detection reagent. Results show that the assay sensitivity is improved with the HRP/Ab:GNP reagent over a colloidal gold reagent, Ab:GNP, and that the reagent can be dried down and reconstituted using the liquid sample alone. Results also indicate that these reagents can be multiplexed.

While the following examples reflect the detection of antigens in stool sample using antigen-specific antibodies, the contents of the disclosure can be used for use in immunoassays for any antigen for which an antibody can be raised or engineered. The antibodies described herein may be replaced with the appropriate antigen-specific antibodies for use in detecting antigens in a variety of sample types that may be processed in accordance with procedures either known the art or antigen specific (e.g., urine samples, fecal samples, blood, serum or plasma sample, or other samples typically and not typically suitable for immunoassays) to the extent the antigen can be solubilized and applied to the device of the disclosure.

### Materials and Methods

The following describes procedures to prepare HRP and an antibody passively coated on a gold nanoparticle (HRP/Ab:GNP), but not the label passively coated on the bold nanoparticle (Ab:GNP), for detection reagents and an antibody passively coated on a latex particle for a capture reagents, all of which are to be used on a SNAP® reversible flow assay device (see e.g., U.S. Pat. No. 5,726,010).

### Preparation of Detection Reagents

### HRP/Anti-Hookworm ADX-12 Ab:GNP

25 ml of naked colloidal gold (40 nm, OD15, from Bioassay Works Inc.) was adjusted pH 7.4 at 10 mM with a concentrated HEPES buffer (1 M at pH7.4). In a separate tube, 0.103 ml of ADX-12 mAb (in-house anti-Hookworm antibody) (7.3 mg/ml in phosphate-buffered saline (PBS)) was mixed with 0.198 ml of HRP (7.6 mg/ml in PBS). The pH-adjusted colloidal gold was added to the ADX-12 antibody-HRP solution and mixed for 3 hr at 4° C. on rotating mixer. 1.25 ml of bovine serum albumin (BSA) (10%, w/v) was mixed with the coated gold suspension to final BSA at concentration as 0.5% and then incubated on a rotating mixer at 4° C. for overnight. The particles were centrifuged down at 15000×g for 15 min and the supernatant was removed. The gold particle pellet was re-suspended into 25 mL of borate buffer (10 mM at pH9 with 0.05% Tween). The coated particles were washed twice with the borate buffer by centrifuge. The washed particles were resuspended into 5.5 mL a conjugate buffer (borate 5 mM pH8.0 with 1% BSA, 20% sucrose, +5% trehalose). The gold particles concentration was determined as optical density (OD) at 530 nm.

### HRP/Anti-Roundworm ADX-10 Ab:GNP

15 ml of naked colloidal gold (40 nm, OD15, from Bioassay Works Inc.) was adjusted pH 7.4 at 10 mM with a concentrated HEPES buffer (1 M at pH7.4). In a separate tube, 0.07 ml of ADX-10 mAb (in-house anti-Roundworm antibody) (7.5 mg/ml in PBS) was mixed with 0.118 ml of HRP (7.6 mg/ml in PBS). The pH-adjusted colloidal gold was added to the ADX-10 Ab-HRP solution and mixed for 3 hr at 4° C. on rotating mixer. 0.75 ml of BSA (10%, w/v) was mixed with the coated gold suspension to final BSA at concentration as 0.5% and then incubated on a rotating mixer at 4° C. for overnight. The particles were centrifuged down at 15000×g for 15 min and the supernatant was removed. The gold particle pellet was re-suspended into 5 mL of borate buffer (10 mM at pH9 with 0.05% Tween). The coated particles were washed twice with the borate buffer by centrifuge. The washed particles were resuspended into 2 mL a conjugate buffer (borate 5 mM pH8.0 with 1% BSA, 20% sucrose, +5% trehalose). The gold particles concentration was determined as OD at 530 nm.

### HRP/Anti-Whipworm ADX-14 Ab:GNP

25 ml of naked colloidal gold (40 nm, OD15, from Bioassay Works Inc.) was adjusted pH 7.4 at 10 mM with a concentrated HEPES buffer (1 M at pH7.4). In a separate tube, 0.07 ml of ADX-14 mAb (in-house anti-Whipworm antibody) (6.4 mg/ml in PBS) was mixed with 0.118 ml of HRP (7.6 mg/ml in PBS). The pH-adjusted colloidal gold was added to the ADX-14 Ab-HRP solution and mix for 3 hr at 4° C. on rotating mixer. 1.25 ml of BSA (10%, w/v) was mixed with the coated gold suspension to final BSA at concentration as 0.5% and then incubated on a rotating mixer at 4° ° C. for overnight. The particles were centrifuged down at 15000×g for 15 min and the supernatant was removed. The gold particle pellet was re-suspended into 5 mL of borate buffer pH9 w/0.05% Tween. The coated particles were washed twice with the borate buffer by centrifuge. The washed particles were resuspended into 2 mL a conjugate buffer (borate 5 mM pH8.0 with 1% BSA, 20% sucrose, +5% trehalose). The gold particles concentration was determined as OD at 530 nm.

### Anti-Hookworm ADX-12 Ab:GNP

26.7 ml of gold nanoparticles (40 nm from Bioassay Works) was adjusted to pH 7.4 with concentrated HEPES buffer at final concentration 10 mM. To the pH adjusted gold particles, 0.3 ml of ADX-12 mAb (6.77 mg/ml) was added dropwise and incubated for one hour at room temperature on rotating mixer. 1.5 ml of BSA (100 mg/mL) was added and kept at 4° ° C. for overnight. The particles were centrifuged at 15000×g for 15 min and the pellet was re-suspended into 15 ml of borate buffer (10 mM at pH8.5 with 0.5% BSA and 0.05% Tween 20). The particles were centrifuged at 15000×g for 20 min and the pellet was re-suspended into 5 ml borate storage buffer (10 mM at pH8.5 with 0.5% BSA). The gold particle concentration was determined based on OD at 530 nm.

### Preparation of Capture Reagents

### Anti-Hookworm RDX-17 Ab Passively Coated on Latex Particles

RDX-17 mAb (IDEXX in-house anti-Hookworm antibody) was dialyzed in 0.1M bicarbonate buffer pH 9.5 three times and final concentration was determined by BCA Protein Assay kit (Thermo Fischer). Next, 1 mL latex particles (0.5 μm size, 5%, w/v %, purchased from Spherotech) was centrifuged at 20000×g rpm for 7 min and then the pellet was re-suspended into bicarbonate buffer (0.1 M at pH9.5). The particles were sequentially sonicated, re-suspended, and centrifuged again. The particle pellet was then re-suspended in 0.1M bicarbonate buffer and 0.203 ml of dialyzed RDX-17 mAb was added as final concentration at 2 mg/ml. The mixture was sonicated and incubated by mixing end-over-end for 1.5 hours at room temperature. The mixture was then centrifuged at 14500 rpm for 7 min. The supernatant was collected and the residual protein in the supernatant was measured by a BCA kit to determine the antibody coating efficiency. The pellet was re-suspended into in bicarbonate buffer with 2.5% BSA (0.1 M at pH 9.5) by sonication and then incubated by mixing end-over-end at room temperature for 1 hr. The particles were centrifuged and the supernatant was removed. The pellet was re-suspended into 1 ml Tris buffer (10 mM pH8.0) by sonication. The pellet containing the final antibody coated latex particles was centrifuged and re-suspended again into 1 ml storage buffer (10 mM Tris, pH8.0+2.5% sucrose+Tween 0.05%). The particle concentration was determined by adding 5 ul of the particle in 995 ul of deionized-water, and measure OD at 650 nm.

### Anti-Roundworm ADX-5 Ab Passively Coated on Latex Particles

2 ml of Latex particles (0.5 μm size, 5%, w/v %, purchased from Spherotech) was centrifuged (14500 rpm 7 min) and re-suspended (in 2 ml phosphate buffer of 10 mM at pH7.4) two times. ADX-5 mAb (IDEXX in-house anti-Roundworm antibody) was prepared in coating solution of phosphate buffer (10 mM at pH7.4) as final concentration at 1 mg/ml. Next, 3.146 ml of ADX-5 solution was added into the latex particle pellet, then the mixture was sonicated in monodispersed, and rotated end-to-end for 1.5 hours at 25° C. The mixture was centrifuged at 20000×g for 7 min. The supernatant was collected and the residual protein in the supernatant was measured by BCA kit to determine the antibody coating efficiency. The particle pellet was resuspended into 2.5% BSA in phosphate buffer (10 mM at pH7.4) and incubated at 25 C for 1.5 hours. The particles were centrifuged and the supernatant was removed. The pellet was resuspended into 10 mM Tris buffer at pH8.0. The pellet containing the final antibody coated latex particles was centrifuged and re-suspended again a storage buffer (10 mM Tris at pH8.0, +2.5% sucrose+Tween 0.05%). The particle concentration was determined by adding 5 ul of the particle in 995 ul of deionized-water, and measure OD at 650 nm.

### Anti-Whipworm ADX-6 Ab Passively Coated on Latex Particles

2 ml of Latex particles (0.5 μm size, 5%, w/v %, purchased from Spherotech) was centrifuged (14500 rpm 7 min) and re-suspended (in 2 ml phosphate buffer of 10 mM at pH7.4) two times. ADX-5 mAb (IDEXX in-house anti-Whipworm antibody) was prepared in coating solution of phosphate buffer (10 mM at pH7.4) as final concentration at 1 mg/ml. Next, 3.146 ml of ADX-5 solution was added into the latex particle pellet, then the mixture was sonicated in monodispersed, and rotated end-to-end for 1.5 hours at 25° ° C. The mixture was centrifuged at 20000×g for 7 min. The supernatant was collected and the residual protein in the supernatant was measured by BCA kit to determine the antibody coating efficiency. The particle pellet was resuspended into 2.5% BSA in phosphate buffer (10 mM at pH7.4) and incubated at 25 C for 1.5 hours. The particles were centrifuged and the supernatant was removed. The pellet was resuspended into 10 mM Tris buffer at pH8.0. The pellet containing the final antibody coated latex particles was centrifuged and re-suspended again a storage buffer (10 mM Tris at pH8.0, +2.5% sucrose+Tween 0.05%). The particle concentration was determined by adding 5 ul of the particle in 995 ul of deionized-water, and measure OD at 650 nm.

### Example 1: HRP/Ab:GNP Function on SNAP® Device

A sintered matrix from a SNAP® device was prepared by coating a reagent zone on the matrix (in an area between a designated detection zone and the sample application zone) with a blocking buffer to inhibit chemical interaction between the HRP/Ab:GNP and the matrix. The detection zone included the appropriate capture reagent as described above. The blocking buffer was prepared by combining Boric Acid (0.077 g), Bovine Serum Albumin (BSA), and Polyvinylpyrrolidone (PVP) powder (MW 10,000) (Sigma Aldrich) (2.5 g) in 200 ml deionized water. TWEEN-20 (0.5 mL) was then added and the solution was stirred at room temperature for about 30 minutes to ensure all components were dissolved. pH was adjusted to 8.03 with NaOH and additional water was added to a final volume of 250 mL. The solution was vacuum filtered and refrigerated.

After addition of the blocking buffer to the matrix, the matrix was allowed to dry and was stored in a sealed for bag with desiccant until needed.

HRP/Ab:GNP solution was spotted/deposited directly onto the reagent zone at a concentration of approximately 4 OD in Tris spotting buffer (50 mM Tris, 2.5% Sucrose, 0.05% Tween-20). The volume of the spot was 1 ul. The spotted strip was allowed to dry for 10 minutes at 35° C. before being installed into a SNAP® device containing the absorbent block, wash and substrate wicks as well as a base containing wash and TMB (tetramethylbenzidine) substrate. The device was activated to allow the wash, and then the TMB to flow across the device. It was observed that after 5 minutes the TMB was being oxidized and blue color was becoming visible, with continued color development until the reaction was ended at 12 minutes with the addition of sodium azide.

### Example 2: Detection of Antigen with HRP/Ab:GNP Showed Higher Sensitivity and Specificity Over Ab:GNP

HRP/Anti-Hookworm Ab:GNP detection reagents were used in an experiment to demonstrate the sensitivity increase over Anti-Hookworm Ab:GNP when used as the detection reagent in a SNAP® assay. SNAP® canine matrix was spotted with anti-Hookworm antibody coated latex particles (Hookworm Ab and rAg were used as a model assay). The HRP/Anti-Hookworm Ab:GNP detection reagent was diluted to a concentration of 4OD when mixed with the sample. There were two samples used in this experiment, which were comprised of a sample diluent (0.05M Tris, 0.05% Tween-20, 0.45% Proclin 150, 0.001M EDTA, 40% FBS) and Hookworm recombinant antigen (rAg) (in-house). The negative sample was comprised of sample diluent only and the positive sample was comprised of sample diluent and Hookworm rAg at a concentration of 180 ng/ml.

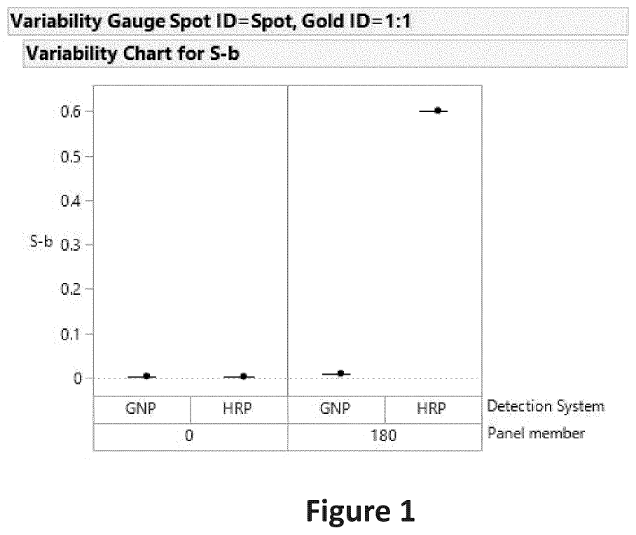

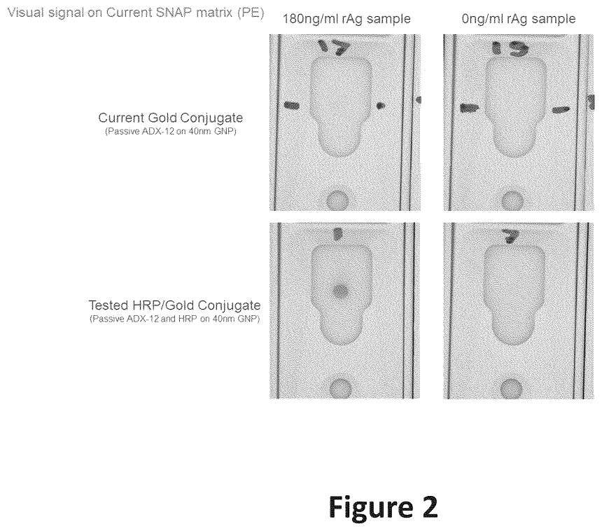

The HRP/Anti-Hookworm Ab:GNP detection reagent was mixed with the sample immediately before adding it to the sample cup of the SNAP® device. The sample mixture was allowed to flow across the membrane until it reached the activation circle, and the SNAP® device was activated. Upon activation a timer was set to 10 minutes to time the assay. At ten minutes the reaction was stopped with 3 drops of sodium azide. A spectrophotometer was used to measure the density of the assay spot as well as the background density of the membrane. The background density was subtracted from the assay spot density to calculate a sample-background (S-B). This data was plotted in a variability chart for analysis (FIG. 1). Images of the visual results from the testing were captured as well (FIG. 2). The continuous and categorical data show that there is a significant increase in sensitivity with the HRP/Anti-Hookworm Ab:GNP detection reagent over the Anti-Hookworm Ab:GNP detection reagent. The results show that there was no decrease in specificity using the HRP/Ab:GNP reagent.

### Example 3: HRP/Ab:GNP in Dried Down State Showed No Change in Sensitivity and Specificity Over Liquid State

To understand if drying the detection reagent has an impact on its performance, the HRP/Anti-Hookworm:GNP was dried into sample tubes before being used to test Hookworm recombinant Antigen (rAg) samples in a SNAP® device. A volume of the HRP/Anti-Hookworm:GNP detection reagent, sufficient to run one SNAP® test, was dispensed into a 1.5 ml microcentrifuge tube and dried under vacuum to remove all water. The tubes were stored overnight in a desiccated foil pouch at room temperature before testing.

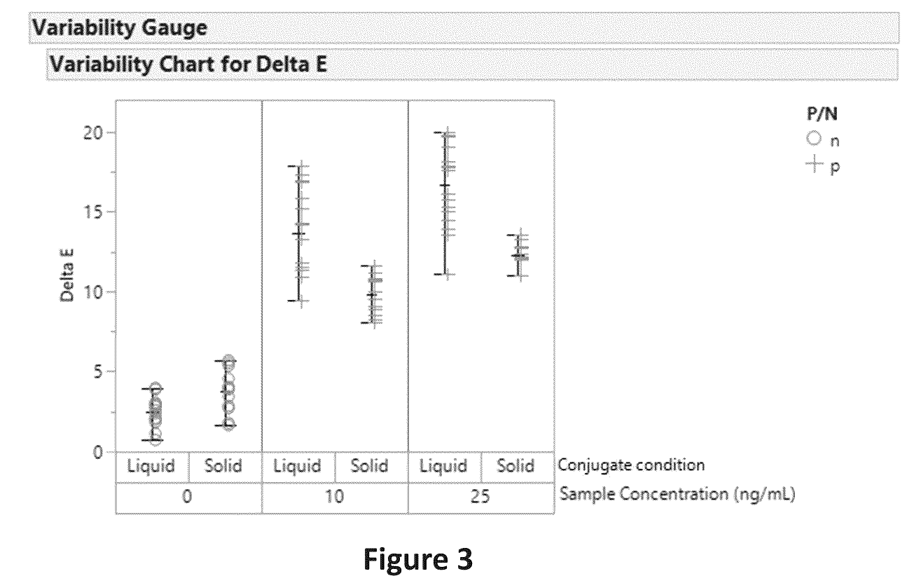

The functionality of the dried conjugates was assessed by comparing the assay signal produced when testing rAg samples with either the dried detection reagent or detection reagent that had not been dried. An appropriate volume of sample was added to the dried detection reagent tube and the mixture was vortexed until it was homogeneous. The contents of the tube were dispensed into the SNAP® device sample cup and the sample front allowed to flow across the matrix to the activation circle before the device was activated. Upon activation, a timer was set to 10 minutes to time the assay. At ten minutes, the reaction was stopped with 3 drops of sodium azide. A spectrophotometer was used to measure the density of the assay spot as well as the background density of the membrane. The background density was subtracted from the assay spot density to calculate a sample-background (S-B). This data was plotted in a variability chart for analysis (FIG. 3). The testing showed that there was no significant negative impact on assay signal when the HRP/Anti-Hookworm:GNP is dried and reconstituted before use.

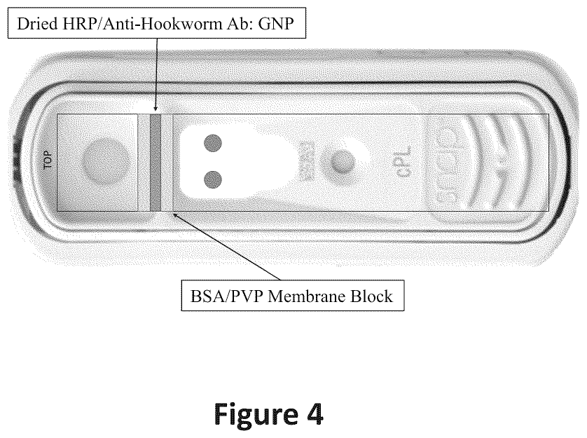

The final embodiment of this detection reagent is to be dried onto the lateral flow membrane to reduce the complexity of the SNAP® assay as well as potentially increase shelf life of the reagent. To demonstrate the potential for dried storage, the HRP/Anti-Hookworm:GNP was striped and air dried onto canine sintered matrix in a location that facilitated its rehydration by the sample as it flowed from the SNAP® sample cup to the activation circle (FIG. 4). The detection reagent was diluted and dispensed in a continuous line across the membrane as shown in FIG. 4. A blocking buffer was dispensed, and dried, onto the detection membrane prior to dispensing reagent.

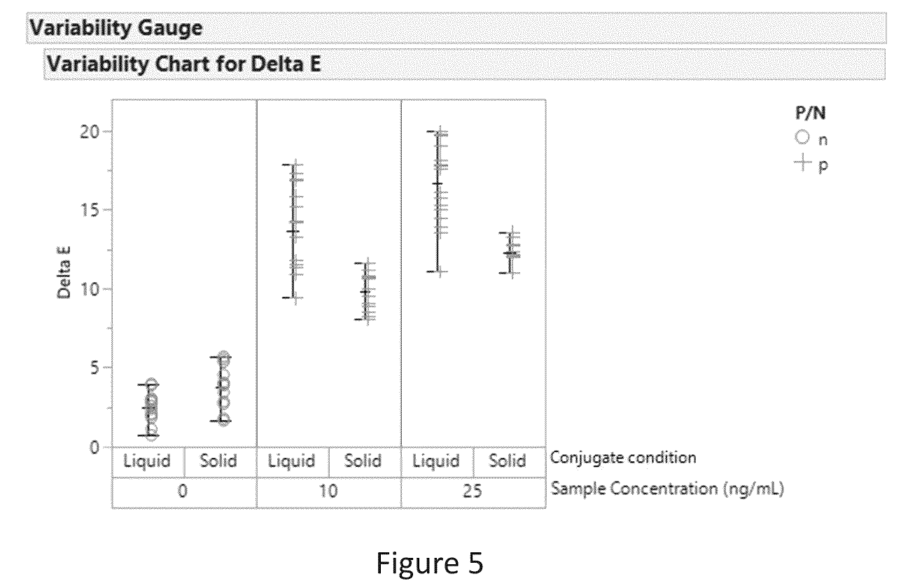

Recombinant Hookworm samples were used to test the functionality of the dried vs liquid HRP/Anti-Hookworm:GNP. The assay was run in liquid form by combining HRP/anti-Hookworm:GNP with the sample diluent containing the recombinant hookworm antigen. The sample/rAg mixture was then dispensed into the sample cup. The dried conjugate devices were run by dispensing the sample diluent containing the recombinant hookworm antigen directly into the sample cup of the SNAP® device. For both assays, the sample was allowed to flow across the membrane until it reached the activation circle, and the SNAP® device was activated. Upon activation a timer was set to 10 minutes to time the assay. At ten minutes, the reaction was stopped with 3 drops of sodium azide. A spectrophotometer was used to measure the density of the assay spot as well as the background density of the membrane. The background density was subtracted from the assay spot density to calculate a sample-background (S-B). The data was plotted in a variability chart for analysis (FIG. 5). This experiment demonstrated that the HRP/Anti-Hookworm:GNP detection reagent can be successfully dried and resuspended from a solid phase matrix without a negative impact on assay functionality.

### Example 4: HRP/Ab:GNP was Capable of Multiplex Immunoassay Detection

To demonstrate the multiplex capability of the HRP/Ab:GNP detection reagent, two additional antibodies were utilized. This testing event shows the functionality of a multiplex of anti-Hookworm, anti-Roundworm and anti-Whipworm HRP/Ab:GNP detection reagents. Each antibody was attached to the gold nanoparticle with HRP in the same fashion as the anti-Hookworm antibody shown in the preceding descriptions. The preparation of the SNAP® devices was identical to that described above in Example 3, except all three HRP/Ab:GNP detection reagents were dried onto the membrane together. In addition, the appropriate anti-Hookworm, Roundworm and Whipworm capture reagents were deposited onto the membrane, i.e., spots.

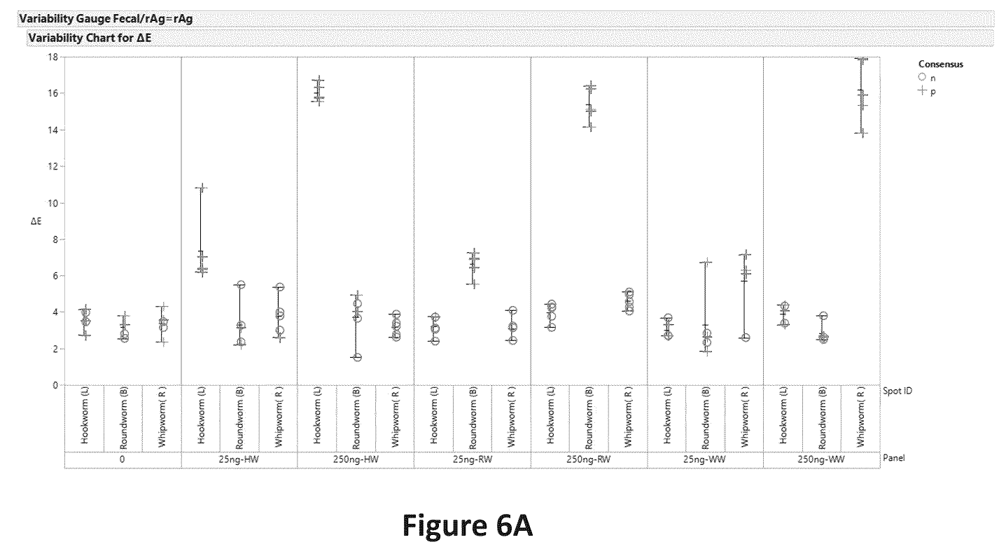

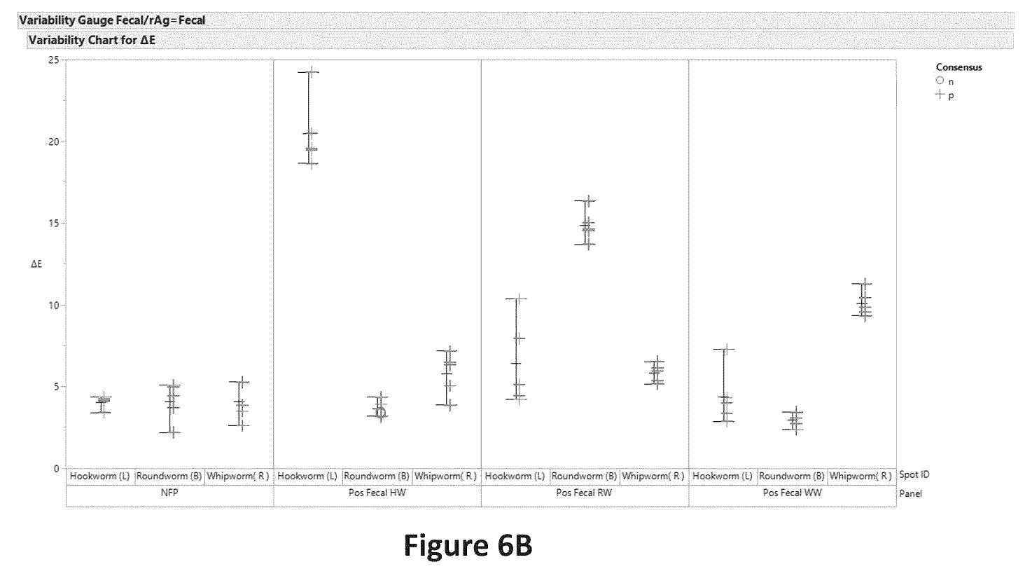

For this testing both rAg samples as well as fecal extract samples were used. The devices were run by dispensing the sample directly into the sample cup of the SNAP® device. The sample was allowed to flow across the membrane until it reached the activation circle, and the SNAP® device was activated. Upon activation, a timer was set to 10 minutes to time the assay. At ten minutes, the reaction was stopped with 3 drops of sodium azide. A spectrophotometer was used to measure the density of the assay spot as well as the background density of the membrane. The background density was subtracted from the assay spot density to calculate a sample-background (S-B). The data was plotted in a variability chart for analysis (FIGS. 6A and 6B).

What is claimed is:

1. An immunoassay device for determining a first analyte in a sample, comprising
a membrane that supports lateral flow of a liquid, and
a dried first detection reagent comprising a first gold nanoparticle having bound thereto (i) a first binding partner for the first analyte, and (ii) a first label, wherein the dried first detection reagent is dried on the membrane and solubilizable by a liquid.

2. The immunoassay device of claim 1, wherein the membrane comprises a sample application zone and a detection zone, and the dried first detection reagent is dried in the sample application zone.

3. The immunoassay device of claim 1, wherein the membrane comprises a sample application zone, a detection zone and a reagent zone between the sample application zone and the detection zone, wherein the dried first detection reagent is dried in the reagent zone.

4. The immunoassay device of claim 2, further comprising a first immobilized capture reagent in the detection zone comprising a second binding partner for the first analyte or a first analyte analog, wherein the second binding partner is non-diffusively bound to the membrane.

5. The immunoassay device of claim 3, further comprising a first immobilized capture reagent in the detection zone comprising a second binding partner for the first analyte or a first analyte analog, wherein the second binding partner is non-diffusively bound to the membrane.

6. The immunoassay device of claim 3, wherein the reagent zone is coated to prevent or inhibit chemical interaction between the dried first detection reagent and the membrane.

7. The immunoassay device of claim 1, wherein the first label is an enzyme and the device further comprises a substrate for the enzyme.

8. The immunoassay device of claim 7, wherein the enzyme is horseradish peroxidase (HRP).

9. The immunoassay device of claim 1, wherein the first label is fluorescent or chemiluminescent.

10. The immunoassay device of claim 4, wherein the first and/or second binding partner for the first analyte is an anti-analyte antibody.

11. The immunoassay device of claim 1, further comprising a housing supporting the membrane and comprising a container comprising a wash reagent.

12. The immunoassay device of claim 7, further comprising a housing supporting the membrane and comprising a container comprising a wash reagent and a container comprising the substrate.

13. A storage stable device comprising the immunoassay device of claim 1, wherein the immunoassay device has a shelf life of over 18 months at room temperature.

14. The immunoassay device of claim 1, further comprising
a sample application zone, a detection zone and, optionally, a reagent zone between the sample application zone and the detection zone, wherein the dried first detection reagent is dried in the sample application zone or the reagent zone, if present.
a dried second detection reagent in the sample application zone or the reagent zone, if present, comprising a second gold nanoparticle having bound thereto (i) a third binding partner for a second analyte, and (ii) a second label, wherein the dried second detection reagent is solulizable by a liquid, and
a second immobilized capture reagent in the detection zone comprising a fourth binding partner for the second analyte,
wherein the first gold nanoparticle and the second gold nanoparticle are the same, and the first label and the second label may be the same or different.

15. The immunoassay device of claim 14, wherein the first label and the second label are the same, and the first immobilized capture reagent and the second immobilized capture reagent are immobilized in distinct regions of the detection zone.

16. A method of determining a presence or amount of an analyte in a sample, the method comprising,
adding a sample suspected of containing the analyte to the sample application zone of the immunoassay device of claim 3,
allowing the sample to migrate by lateral flow through the reagent zone and the detection zone; and
detecting a presence or amount of the first label in the detection zone, wherein the presence or amount of the first label provides a signal associated with the presence or amount of the analyte in the sample.

17. The method of claim 16, wherein the reagent zone is coated to prevent or inhibit chemical interaction between the dried first detection reagent and the membrane.

18. The method of claim 16, wherein the immunoassay device further comprises a housing supporting the membrane and comprising a container comprising a wash reagent, wherein the method comprising washing the detection zone with the wash reagent.

19. The method of claim 16, wherein the immunoassay device further comprises a housing supporting the membrane and comprising a container comprising a wash reagent and a container comprising a substrate of the first label, wherein the method comprises (i) washing the detection zone by adding the wash reagent to the membrane and washing the detection zone with the wash reagent, and (2) adding the substrate to the detection zone.
